(19) United States
(12) Patent Application Publication
(10) Pub. No.: US 2022/0142565 A1
(43) Pub. Date: May 12, 2022

(54) COMPOSITION FOR MEASURING MEDICATION COMPLIANCE AND METHOD THEREOF

(71) Applicants: UNIVERSITE GRENOBLE ALPES, Saint Martin D'Heres (FR); INSTITUT POLYTECHNIQUE DE GRENOBLE, Grenoble (FR); CENTRE NATIONAL DE LA RECHERCHE SCIENTIFIQUE, Paris (FR); CENTRE HOSPITALIER UNIVERSITAIRE GRENOBLE ALPES, La Tronche (FR)

(72) Inventors: Jean-Pierre ALCARAZ, Pontcharra (FR); Donald MARTIN, Gieres (FR); Philippe CINQUIN, Saint Nazaire Les Eymes (FR)

(21) Appl. No.: 17/437,380

(22) PCT Filed: Mar. 4, 2020
(86) PCT No.: PCT/EP2020/055736

§ 371 Date: Sep. 8, 2021

(30) Foreign Application Priority Data
Mar. 8, 2019 (FR) 1902393

(51) Int. Cl. A61B 5/00; A61K 49/00; A61B 5/145; A61B 5/07

(52) U.S. Cl. CPC A61B 5/4833; A61K 49/0004; A61B 5/076; A61B 5/14503; A61B 5/14546

(57) ABSTRACT

Therapeutic compliance and more particularly a composition having: a drug substance, selected from an active ingredient and a placebo, and a detection agent. The drug substance being different from the detection agent. The composition being configured such that the detection agent allows to indicate an ingestion of the drug substance. The composition is such that the detection agent includes a hydride that will dissolve upon contact with an aqueous medium in a human or non-human animal body, releasing dihydrogen.

### TECHNICAL FIELD

The invention relates to the field of drug treatment compliance. It finds a particularly advantageous application in measuring compliance with treatments for chronic diseases.

### PRIOR ART

Therapeutic treatments, especially in the context of chronic diseases, may require the controlled and regular intake of many drugs. This necessity can make treatment difficult to follow, especially for patients with cognitive impairment.

Lack of therapeutic compliance is the root cause of many health problems for patients. For example, poor compliance to anticoagulant treatments is the cause of very many vascular accidents which can lead to death, due to incorrectly taking the treatment, either by excess when the patient takes his treatment several times, or by default when the patient forgets to take his treatment.

There are tools to manage therapeutic compliance. In particular, daily, weekly or specific, manual or electronic pill organisers can be used. However, these pill organiser systems do not indicate actual ingestion of the treatment by the patient and are therefore limited to measuring actual therapeutic compliance to without the additional need for sampling, such as blood or urine samples.

Among the existing solutions, one consists in providing a composition comprising at least one drug substance, selected from at least one active ingredient and a placebo, and at least one detection agent, the drug substance being different from the detection agent, the composition being configured such that the detection agent allows to indicate the ingestion of the drug substance and allows to measure the therapeutic compliance.

Such a composition is known from document US 2008/0213904 A1. This document describes the optical detection of the delivery of an active ingredient by means of a marker, for example a fluorophore. After ingestion of the composition comprising the marker and the active ingredient, said marker can be detected by an optical imaging device external to the patient. Advantageously, a combination of markers can be used in order to create an optical signature specific to at least one active ingredient, so that it is possible to determine which ingredient or which active ingredients have been ingested. However, due to the limited penetration through the body of light in the visible wavelengths, the optical imaging device must be disposed on preferential sites, such as the retina or else areas of the body where the skin thickness is thin.

Analysing a set of optical signatures can require complex equipment to probe a wide wavelength domain. Furthermore, it is necessary for the marker, or the combination of markers, to be diffused in sufficient amount to said preferential site, which may delay the detection of ingestion of the composition. However, real-time detection has the advantage not only of avoiding possible oversights but also of being able to adapt the intake of medications sensitive to chronopharmacology.

Moreover, it is known from document U.S. Pat. No. 8,597,186 B2, an ingestible device including at least one active ingredient and at least one ingestion event marker consisting of an ion emission module. The module is activated upon contact with a conductive fluid, such as stomach or intestinal fluid, to emit a signal detectable by a device implanted or disposed on the surface of the patient's body. Said signal, of radiofrequency, magnetic or acoustic nature, can be generic in order to detect the ingestion of any composition of any kind, or specific to a given emission module, which allows for example to determine the active ingredient ingested.

Each module requires a miniaturised electronic equipment potentially specific to each active ingredient administered by ingestion of the device. It is understandable that such a device could lead to an additional cost of production.

The current compositions and methods therefore show limits. In particular, none of them allows compliance measurement which is in real time and at a reduced production cost for the pharmaceutical industry.

In this context, the present invention proposes, according to a first aspect, a composition allowing to overcome at least one of the aforementioned drawbacks.

More particularly, the composition according to the first aspect of the invention aims at allowing the detection of the ingestion of a drug substance in real time, while having an acceptable production cost for the pharmaceutical industry so that the composition is adapted for a very large deployment. It would also be advantageous for such a composition to allow to obtain a signature specific to a class of drug substances, to a particular drug substance, or even to the specific formulation of said drug substance.

The other objects, features and advantages of the present invention will become apparent upon examining the following description and the accompanying drawings. It is understood that other advantages can be incorporated.

### SUMMARY

To achieve at least one of the aforementioned objectives, according to a first aspect, the present invention provides a composition, moreover in accordance with the generic definition given in the preamble above, wherein said at least one detection agent comprises at least one hydride able to be dissolved upon contact with an aqueous medium in the human or animal body, releasing dihydrogen.

According to one embodiment of the first aspect of the invention, the composition consists of at least one drug substance and at least one detection agent, or even of a single drug substance and of a single detection agent, as introduced above.

It should also be noted that the dihydrogen released is in dissolved and/or gas form. Furthermore, the detection agent can consist only of said at least one hydride or comprise additional elements, for example an additive or an excipient.

The release of dihydrogen during the dissolution of said at least one hydride upon contact with an aqueous medium in the human or animal body allows detection of the ingestion of the composition in real time. Furthermore, the production of said at least one detection agent according to the features of the composition previously stated and the following features has a production cost which is acceptable to the pharmaceutical industry. Thus, the composition according to the present invention is adapted for a very wide deployment.

Optionally, the invention may further have at least any one of the following features:
said at least one hydride is formulated so as to release dihydrogen in a specific part of the human or animal body, preferably in the stomach. The composition according to this latter feature constitutes a preferred embodiment of the invention. It has the advantages stated above and is compatible with the following features of the composition;
said at least one detection agent may further comprise bicarbonate formulated so as to release carbon dioxide in a specific part of the human or animal body, preferably in the stomach. Thus, the treatment compliance can be measured by detecting gases released by hydride and bicarbonate, thus enhancing the reliability of the measurement;
the composition may comprise at least one formulating agent of said at least one detection agent, each formulating agent being configured to be degraded in a specific part of the human or animal body, preferably in the stomach, so as to release said at least one detection agent;
the composition may further comprise a formulating agent of said at least one drug substance, the formulating agent being configured to be degraded in a specific part of the human or animal body, preferably in a part of the gastrointestinal tract posterior to the stomach, so as to release said at least one drug substance therein. The formulating agent for the drug substance may be of the same constitution.
each formulating agent can be configured to structure the detection agent and/or the drug substance it formulates. Each formulating agent can more particularly comprise at least one of:
   a coating configured to coat the detection agent and/or the drug substance that it formulates and
   a binder to bind together particles of the detection agent and/or the drug substance that it formulates. Each formulating agent then also plays the role of a structuring agent of the composition. The composition can thus have a structure in successive concentric layers, or in superimposed flat layers. The formulating agent of the drug substance may have the same structuring function as the formulating agent of the detection agent; one can even be confused at least in part with the other. On the contrary, the formulating agent of the drug substance may be of a different constitution, or even have a structuring function which is different than that/those of the formulating agent of the detection agent. For example, the formulating agent of the drug substance can be a binder wherein the drug substance is mixed and the formulating agent of the detection agent can be a coating containing at least the detection agent;
each detection agent and products formed by its dissolution as well as, where appropriate, each formulating agent and products formed by its degradation, are preferably pharmaceutically acceptable. Of course, the drug substance and products possibly formed by its dissolution are pharmaceutically acceptable. Indeed, products resulting from the dissolution of the detection agent, or even from the drug substance, or from the degradation of the formulating agent can be formed which are entirely biocompatible and eliminable by the stool or by the fluids of the body. The pharmaceutical acceptability, or non-toxicity, of any one of the drug substance, the detection agent and the formulating agent, as well as their derivative products, is to be assessed, in particular in terms of limit dose, with regard to the disease to be treated and its actual or potential consequences. The pharmaceutical acceptability, or non-toxicity, of the detection agent is to be assessed with regard to the benefit that the human or animal subject may have from the measurement of therapeutic compliance thanks to the composition according to the invention;
the drug substance can be mixed or juxtaposed directly or indirectly with said at least one detection agent, for example said at least one drug substance is contained in a coating formed at least in part by said at least one detection agent;
said at least one formulating agent is configured such that the release of the hydride and the delivery of the drug substance are simultaneous or delayed in time, preferably the delivery of the active ingredient being carried out subsequently to the release of the hydride. According to this additional feature, the drug substance can be delivered before the release of the dihydrogen, for example in the mouth or the oesophagus, simultaneously with the release of the dihydrogen, preferably taking place in the stomach, or after the release of the dihydrogen, for example in a part of the gastrointestinal tract posterior to the stomach such as the intestine;
said at least one formulating agent of said at least one hydride is configured such that its degradation causes a release of dihydrogen which is uniform or variable overtime. Where appropriate, said at least one formulating agent of said bicarbonate is configured such that its degradation causes a uniform or variable release of carbon dioxide over time. The composition according to one and/or the other of these two additional features allows to control the amount and the duration of the release of dihydrogen and/or carbon dioxide, or even to control the evolution over time of this amount, upon degradation of the formulating agent of said at least one detection agent. Thus, in addition to allowing the detection of the ingestion of the composition comprising at least one drug substance, the release of dihydrogen and/or carbon dioxide constitutes a signature potentially specific to said at least one drug substance and can also allow to know the dose of drug ingested;
when the composition comprises a plurality of formulating agents of said at least one detection agent and/or of said at least one drug substance, the formulating agents of this plurality can be configured with one another. At least two formulating agents of said plurality can more particularly be mixed together, structured in successive concentric layers, or structured in superimposed flat layers. The composition according to this additional feature allows to control the amount and the duration of the release of dihydrogen and/or carbon dioxide, or even to control the evolution over time of this amount, during the degradation of said plurality of formulating agents. Thus, it is understood that it is possible to obtain a multitude of signatures potentially specific to a class of drug substances, a particular drug substance, or even a specific formulation of a drug substance;
each formulating agent can be based on at least one of:
   a material soluble in contact with a medium of determined hydrogen potential (pH),
   a material based on a biodegradable polymer, such as lactic acid polymer,
   a material soluble in contact with an aqueous medium, and
   a gel;
said at least one detection agent is preferably porous. The hydride thus provides an increased contact surface with the aqueous medium for a more efficient release of the dihydrogen. With the same advantage, bicarbonate can also be porous;
Said at least one detection agent, and in particular said at least one hydride, may be in the form of a powder, the particles of which preferably have an average size comprised between 10 nm and 10 μm. Each detection agent thus provides an increased contact surface with the aqueous medium for more efficient release of dihydrogen and/or carbon dioxide. Furthermore, the detection agent thus formed is easy to contain and/or bind;
Said at least one hydride may be based on at least one of silicon hydride, magnesium hydride and calcium hydride;
Said at least one hydride is preferably based on porous silicon. The porosities can be of mesoscopic and/or nanoscopic size. The composition according to this latter feature constitutes a highly preferred embodiment of the invention. It allows to combine the advantages previously stated.

It emerges from the foregoing that the composition can advantageously take many forms corresponding to as many formulations and structuring of the composition.

Another aspect of the present invention relates to a method for measuring therapeutic compliance, the method implementing at least one measurement and communication system.

Said system comprises a dihydrogen sensor, a wireless transmission device and an external wireless reception device. The system may further comprise a carbon dioxide sensor. Each sensor and said transmission device are disposed in the human or animal body, each sensor being operatively connected to the transmission device.

According to this method, after the ingestion of a composition as previously introduced, the dihydrogen and possibly the carbon dioxide released is detected by the sensor of dihydrogen and, where appropriate, of carbon dioxide. A radiofrequency signal indicating this detection is emitted by the wireless transmission device. Said signal is received, as a measurement of therapeutic compliance, by the external wireless receiving device.

For example, a capsule able to be introduced into the digestive system and more particularly into the gastrointestinal tract is known from document WO 2018/032032 A1. Said capsule comprises in particular at least one gas sensor capable of detecting dihydrogen naturally produced in the gastrointestinal tract, and more particularly in the stomach (A human pilot trial of ingestible electronic capsules capable of sensing different gases in the gut, Nature Electronics, vol. 1, pp. 79-87, 2018, doi: 10.1038/s41928-017-0004-x). Such a sensor seems to be able to be used in the measurement and communication system introduced above. In particular, its low detection limit or its sensitivity is adapted for the implementation of the method according to the second aspect of the invention, in the sense that the composition is such that the amount of gas that it allows to release is preferably, or even necessarily, higher than the amount of this gas that is naturally produced at the location of detection.

Moreover, it is known from document US 2017/0058282 A1 and from the document by Mimee and al. (An ingestible bacterial-electronic system to monitor gastrointestinal health, Science, vol. 360 (6391), pp. 915-918, 2018, doi: 10.1126/science.aas9315) a capsule able to be introduced into the digestive system and more particularly into the gastrointestinal tract. Said capsule comprises in particular at least one sensor comprising bacteria modified so as to detect a haemorrhagic effusion in the gastrointestinal tract and produce a signal sensed by an electronic equipment comprised in said capsule. It seems reasonable, in view of the invention according to its first aspect, to consider that this sensor can be adapted to the detection of dihydrogen and/or carbon dioxide in the gastrointestinal tract, and more particularly in the stomach.

Dihydrogen sensors and carbon dioxide sensors adapted to implement the method according to the second aspect of the invention exist. These sensors allow to detect the gas considered, or even to measure the variation in the amount of this gas over time. The choice of a dihydrogen sensor and/or a carbon dioxide sensor from these existing sensors falls within the ordinary skill of the person skilled in the art who will take into account its low and possibly high detection limit, as well as a possible detection threshold to be set as a function of the composition according to the first aspect of the invention, and which, where appropriate, will also take into account its ability to measure the change over time in the amount of gas released.

According to an additional feature of the method introduced above by the present invention, the dihydrogen sensor, and optionally the carbon dioxide sensor, is/are preferably placed in a specific part of the human or animal body, preferably in the stomach. Since the sensor(s) and the wireless transmission device are operatively connected, they preferentially form a module attached to the wall of the stomach by an anchor. The sensor(s) and said transmission device can also be movable by being integrated into a capsule as described in documents WO 2018/032032 A1 and by Mimee and al. previously mentioned.

The method according to the second aspect of the present invention can use many types of sensors and many types of wireless transmission devices, which are known from the prior art or to come.

A miniaturised implantable system able to be fixed to a wall of the stomach of a patient by an anchor and allowing radiofrequency communication with a device located outside the body of a patient, is in particular known from document FR 3059558 A1.

The external wireless reception device is worn by the patient, for example said external device is a smartphone, a digital tablet, a connected watch, a dedicated apparatus or else a module integrated into or connected to a mobile phone or a digital tablet. In addition, at least one second external device, for example located in the patient's home or in a doctors office, can be comprised in the measurement and communication system and implemented by the method according to the second aspect of the present invention.
The method described by the invention can advantageously be used for measuring therapeutic compliance in several ways:
the dihydrogen or carbon dioxide sensor can be configured, not only to detect the presence of dihydrogen or carbon dioxide, respectively, in said specific part of the human or animal body, but also to measure the amount of dihydrogen or carbon dioxide, respectively, present in said specific part of the human or animal body, or even to measure the evolution over time of this amount. Thus, a multitude of diverse and varied signatures are detectable by the measurement and communication system, said signatures being potentially specific to a class of drug substances, a particular drug substance, or even a specific formulation of a drug substance. According to this arrangement, the measurement of the therapeutic compliance can be carried out for a multitude of drug substances or compositions, identical or different from one another, taken successively or even concomitantly;
the signal emitted by the wireless transmission device to the external wireless reception device can be recorded in a memory, preferably non-volatile memory, of said external device in order to constitute a history of the therapeutic compliance to at least one treatment. Said history can subsequently and if necessary be relayed to a second external device, which can be consulted for example by the medical staff. The history of therapeutic compliance allows the patient and/or the medical staff to follow the drug treatment a posteriori. The medical staff can thus advantageously adapt the treatment and follow the patient within the framework of personalised medicine;
the method may further comprise a step of reminding said patient of the need to ingest at least one composition as introduced previously. More particularly, a reminder signal can be emitted by the external device to request, from the patient, the ingestion of at least one composition, at a defined time. The ingestion can then be checked as described above. The reminder signal can be a sound, light signal, a vibration or a written message, for example of the SMS type. The emission of the reminder signal can be defined temporally with respect to the initial or previous ingestion of said at least one composition the therapeutic compliance of which has been measured by implementing the method or can be predetermined temporally to request a medication intake at specific times. Furthermore, in either of these two cases, if the ingestion of the composition is not detected within a given time after the last reminder, at least a second reminder signal may be emitted. The method according to this additional feature has the advantage not only of avoiding possible omissions but also of adapting the intake of medications which are sensitive to chronopharmacology.

Said method therefore advantageously offers easier and soothing compliance for the patient who no longer runs the risk of forgetting to take said at least one composition. Compliance and therapeutic adherence are thus promoted by the influence of said method on the patient's behaviour with respect to his treatment and its effects.

### BRIEF DESCRIPTION OF THE FIGURES

The purposes, objects, as well as the features and advantages of the invention will become more apparent from the detailed description of particular embodiments of the latter which are illustrated by the following accompanying drawings wherein:

FIGS. 1A to 1E illustrate different embodiments of the composition;

FIGS. 2A to 2E illustrate different modes of formulation/structuring of said at least one detection agent;

FIGS. 3A to 3E each illustrate a mode of release of dihydrogen as a function of time t, each mode relating to the formulation/structuring illustrated in the corresponding figure from FIGS. 2A to 2E;

FIG. 4 illustrates a transparent view of some parts of a human body and the relative placement of some devices of the measurement and communication system allowing the implementation of the method according to the second aspect of the invention;

FIG. 5 schematically illustrates the hydride dissolution reaction in aqueous medium; and

FIG. 6 illustrates some steps of the method for measuring the compliance according to an embodiment of the second aspect of the invention.

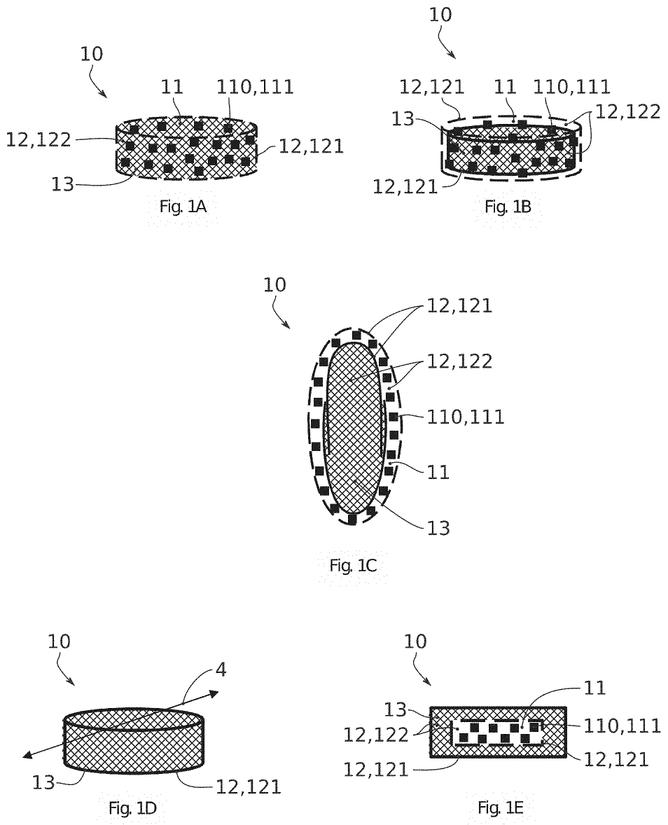

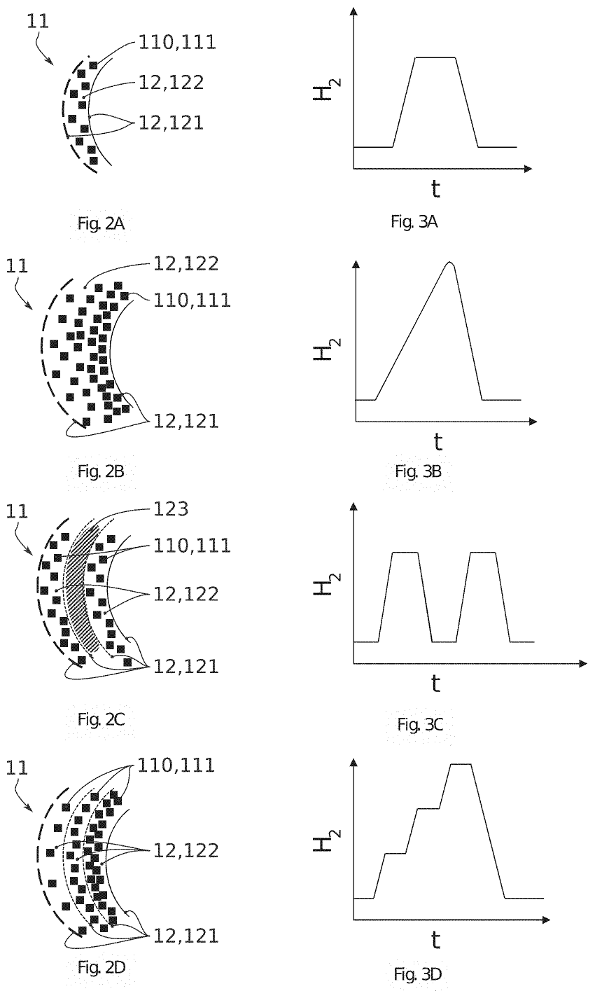

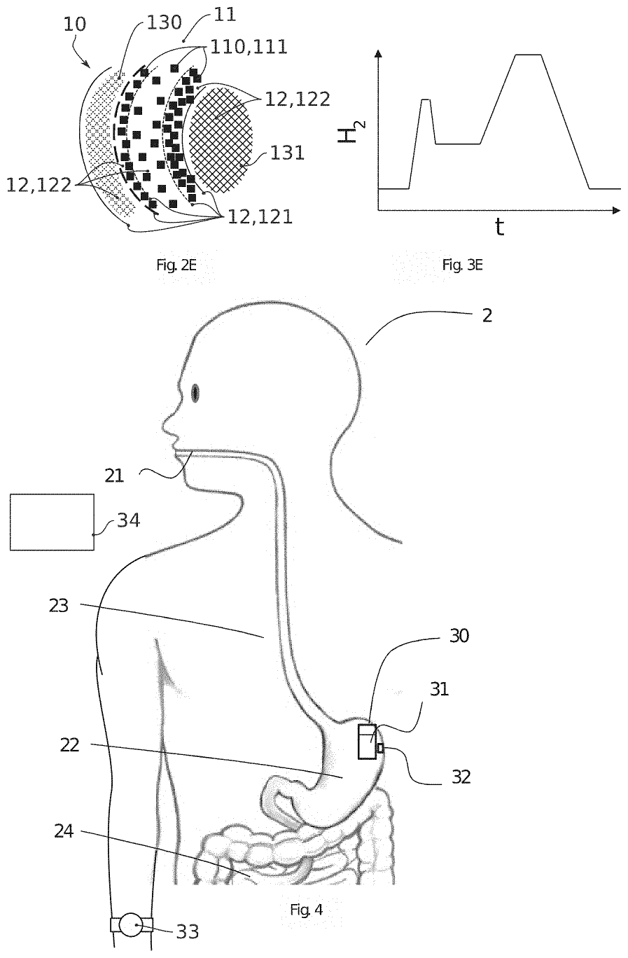

The drawings are given by way of example and are not limiting of the invention. They constitute schematic principle representations intended to facilitate understanding of the invention and are not necessarily on the scale of practical applications. In particular, the respective sizes of the different embodiments illustrated in FIGS. 1A to 1E and 2A to 2E are not intended to be compared with each other.

Expressions such as “equal, lower, higher” are understood to mean comparisons that can accommodate some tolerances, in particular depending on the magnitude of the compared values and the measurement uncertainties. Values substantially equal, lower or higher are within the scope of the interpretation of the invention.

“Detection agent” here means a compound or a formulation comprising at least one hydride, the dissolution of which in an aqueous medium results in the release of dihydrogen.

“Formulation” means the determination of the relative amounts of various elements entering into a composition, or else the determination of the relative arrangement of these various elements relative to each other. A formulating agent actively participates in this determination, at least as part of the composition, or else as a structuring element of the composition. In the latter case, the formulating agent is more particularly a structuring agent.

It is specified that, in the context of the present invention, the term “successive” or “juxtaposed” or their equivalents do not necessarily mean “in contact with”. Thus, two “successive” or “superimposed” layers are not necessarily in contact.

“Dihydrogen sensor” means a device capable of detecting dihydrogen, more particularly for measuring the amount or even for measuring the change in the amount of dihydrogen produced by dissolving said at least one hydride, said device being disposed in a specific part of the human or animal body, preferably in the stomach.

“Carbon dioxide sensor” means a device capable of detecting carbon dioxide, more particularly for measuring the amount or even for measuring the change in the amount of carbon dioxide produced by dissolving the bicarbonate optionally comprised in the composition according to the first aspect of the invention, said device being disposed in a specific part of the human or animal body, preferably in the stomach.

“Wireless transmission device” means a device capable of transmitting without a wired connection a radiofrequency signal signifying the detection, more particularly the measurement of the amount, or even the evolution of the amount of dihydrogen, carried out by the dihydrogen sensor and possibly by the carbon dioxide sensor.

“External wireless reception device” means a device located outside the human or animal body and capable of receiving, without a wired connection, the radiofrequency signal transmitted by the wireless transmission device.

In the following, particular embodiments are described wherein the drug substance is at least one active ingredient. Note that the following features and benefits also apply in case the drug substance is a placebo. Thus, the drug substance has no therapeutic effect. It is indeed advantageous to use a composition according to the first aspect of the invention and to implement it by a method according to the second aspect of the invention, even when the drug substance has no therapeutic effect, because this will allow both the medical staff and the patient to assess, based on the compliance measurements thus carried out, to judge the relevance of then proceeding with the therapeutic treatment itself, or else to choose the most appropriate therapeutic treatment, if not in medical terms, at least in terms of the patient's ability to monitor the treatment.

Referring to FIGS. 1A to 1E, the invention relates firstly to a composition 10 comprising at least one detection agent 11 comprising a hydride 110.

The hydride 110 is in particular able and intended to be dissolved upon contact with an aqueous medium and thus release dihydrogen.

The detection agent 11 may further comprise a formulating agent 12 of said at least one hydride 110.

The formulating agent 12 which has not been degraded, or before its degradation, can be configured to isolate hydride 110 from an environment of the composition 10. Thus, the formulating agent 12 allows, except in the case of degradation, to preserve the hydride 110 contained in the composition 10 from any contact with the environment, and in particular from any contact with an environment capable of causing the release of dihydrogen, in particular any surrounding aqueous or even humid medium.

The formulating agent 12 according to the invention can be understood as a coating 121 to coat the hydride 110 or particles 111 of the hydride. Alternatively or in addition, the formulating agent 12 according to the invention can be understood as a binder 122 to bind together particles 111 of the hydride.

By the choice of the composition and the thickness of the formulating agent 12, it is possible to control the specific part of the human or animal body 2, and in particular the part of the gastrointestinal tract, where the formulating agent 12 will be degraded. A targeted release of dihydrogen is thus obtained, in particular on at least part of the gastrointestinal tract, preferably in the stomach 22.

By the choice of the composition and the thickness of the binder 122 of the formulating agent 12, it is also possible to control the amount of dihydrogen released and the duration of release of the dihydrogen in at least part of the gastrointestinal tract, preferably in the stomach 22.

The formulating agent 12 can more particularly be configured to be degraded under at least one observable physiological condition specific to a part of the human or animal body 2, and more particularly in at least part of the gastrointestinal tract, preferably in the stomach 22. More particularly still, the formulating agent 12 can be configured to release hydride 110 only when the composition 10 is under at least one specific physiological condition. The formulating agent 12 can indeed be selected to be degraded when placed in a specific environmental condition defined by a physiological parameter or a combination of physiological parameters among which the presence, or else the amount of water, the temperature, the pH, the concentration of inorganic salts, etc., such a physiological parameter or such a combination of physiological parameters being observable in at least one location of the human or animal body 2, or else in at least part of the gastrointestinal tract, preferably in the stomach 22.

The degradation of the formulating agent 12 thus allows the hydride 110 to be contacted with the aqueous medium which constitutes the part of the human or animal body 2 where said specific physiological condition prevails, if applicable. It may indeed be preferable, or even required, depending on the treatment to be followed, for said physiological condition or said combination of physiological conditions to define at least the stomach 22 rather than the mouth 21 or the oesophagus 23 of the human or animal body 2, in particular with regard to the mode of administration by ingestion of the composition 10. Consequently, the release of dihydrogen can be carried out, thanks to the composition 10 according to the invention, preferably in the stomach 22 of the human or animal body 2.

It is the formulation offered by the formulating agent 12 which allows to control, after the ingestion of the composition 10, at least one location where said release will take place, preferably the stomach 22, and at least one of the amount and duration of the release of dihydrogen.

With reference to FIGS. 1A to 1E, the composition 10 further comprises at least one active ingredient 13. The composition may further comprise a formulating agent 12 of said at least one active ingredient 13.

The formulating agent 12 of said at least one active ingredient 13, when it is not degraded, or before its degradation, can be configured in particular to isolate the active ingredient 13 from an environment of the composition 10. Thus, the formulating agent 12 can allow, except in the event of degradation, not to deliver the active ingredient 13 contained in the composition 10 into the environment of the composition.

The formulating agent 12 according to the invention can be understood as a coating 121 for coating the active ingredient 13. Alternatively or in addition, the formulating agent 12 according to the invention can be understood as a binder 122 to bind together particles of the active ingredient 13, in the case where the active ingredient 13 is in the form of a powder.

By the choice of the composition and the thickness of the formulating agent 12, it is possible to control the specific part of the human or animal body 2, and in particular the part of the gastrointestinal tract, where the formulating agent 12 will be degraded. A targeted delivery of the active ingredient 13 is thus obtained, preferably in a part of the gastrointestinal tract posterior to the stomach, such as the intestine 24.

The formulating agent 12 can more particularly be configured to be degraded under at least one observable physiological condition specific to a part of the human or animal body 2, preferably in a part of the gastrointestinal tract posterior to the stomach such as the intestine 24. More particularly still, the formulating agent 12 can be configured to release the active ingredient 13 only when the composition 10 is under at least one specific physiological condition. The formulating agent 12 can indeed be selected to be degraded when placed in a specific environmental condition defined by a physiological parameter or a combination of the physiological parameters described above, said parameter or said combination being observable in at least one location of the human or animal body 2, preferably in a part of the gastrointestinal tract posterior to the stomach such as the intestine 24.

Thus, the composition 10 allows, by degradation of the formulating agent 12 of said at least one detection agent 11 and dissolution of the hydride 110, to release dihydrogen in at least one location of the human or animal body 2, or else in at least part of the gastrointestinal tract, and more particularly in the stomach 22, this part being defined by one or more physiological conditions.

Furthermore, the composition 10 allows, by degradation of the formulating agent 12 of said at least one active ingredient 13, the delivery of the active ingredient in a specific part of the human or animal body 2, preferably in a part of the gastrointestinal tract posterior to the stomach such as the intestine 24, this part being defined by one or more physiological conditions.

It is then understood that the composition 10 comprising at least one formulating agent 12 of said at least one detection agent (11) and at least one formulating agent 12 of said at least one active ingredient 13 allows the release of the hydride 110 and the delivery of the active ingredient 13 simultaneously or delayed in time. The formulating agent 12 of said at least one detection agent 11 and the formulating agent 12 of said at least one active ingredient 13 can be mixed or form a single formulating agent so that said release and said delivery are simultaneous. The formulating agent 12 of said at least one detection agent 11 and the formulating agent 12 of said at least one active ingredient 13 can be juxtaposed so that said release and said delivery are delayed in time. Preferably, the formulating agent 12 of said active ingredient 13 is contained in a coating comprising at least part of said at least one detection agent 11 so that the delivery of the active ingredient 13 is carried out subsequently to the release of the hydride 110. The release of the detection agent 11 and the delivery of the active ingredient can for example be delayed by at least 5 minutes, or even at least 20 minutes, or even at least 30 minutes, or even at least one hour. This delay can in particular be chosen according to the location of the composition in the gastrointestinal tract where it is desired to release the detection agent, for example the stomach, and the location of the composition in the gastrointestinal tract where it is desired to deliver the active ingredient 13, for example the mouth or the intestine.

The formulating agent 12 can be based on at least one of:
a material soluble in contact with a medium of determined hydrogen potential (pH),
a material based on a biodegradable polymer, such as polylactic acid (PLA),
a material soluble in contact with an aqueous medium, and
a gel.

These different materials can have different degradation speeds, possibly under equivalent environmental conditions. It is for example advantageous that different formulas of the formulating agent 12 are based on a material chosen to have a determined degradation speed and in or under a physiological condition defining a specific part of the human or animal body 2, said degradation speed being preferably compatible with the duration of residence of the composition in said specific part of the human or animal body 2.

Furthermore, it is considered that the composition 10 comprises a plurality of formulating agents 12 of said at least one detection agent configured together so as to be degraded differently under the same physiological condition preferentially defining the stomach 22. For example, several formulating agents 12 of said at least one detection agent can be arranged in a configuration in successive concentric layers. More particularly, a first formulating agent 12 comprising a first amount of a first hydride 110 may be coated with a second formulating agent 12 different from the first formulating agent 12 and optionally comprising a second amount of a second hydride. 110. In this case, the first and second hydrides can be different from each other and/or the first and second amounts of hydride can be different from each other. An example of a configuration alternative to a configuration in successive concentric layers may consist in superimposing or juxtaposing layers which are substantially flat with one another. For example, it is further considered that inclusions of a first formulating agent 12 comprising a first amount of a first hydride 110 are bound together by a second formulating agent 12 free of hydride.

As will be described in more detail later with reference to FIGS. 2A to 2E, it is understood, in view of the above stated compositions of the formulating agent 12, that it is sufficient to vary the amount or the relative arrangement of the formulating agent 12 or of a set of formulating agents 12 of said at least one detection agent 11 to vary the amount of dihydrogen and the duration of release of the dihydrogen upon degradation of the detection agent 11.

Thus, the composition 10 allows, by degradation of the formulating agent 12 of said at least one detection agent 11 and dissolution of the hydride 110, to release dihydrogen in a modulated manner, over time, or even along the gastrointestinal tract. Said release is carried out in a determined amount over a controlled duration or in several determined and potentially variable amounts over controlled periods, said one or more periods being preferably compatible with the residence time of the composition 10 in the specific part of the human or animal body 2 when this and only this one is targeted. “Modulated release” means here that the amount of dihydrogen released varies over time simultaneously with the degradation of one or more formulating agent(s) inducing the dissolution of one or more hydride(s). For this purpose, the distribution of the detection agent 11 in the composition can be structured non-uniformly by the formulating agent 12. The formulating agent 12 can be configured such that its degradation and the dissolution of the compound is carried out over a period comprised between 30 seconds and 1 hour, or even between 30 seconds and 30 minutes, preferably between 30 seconds and 10 minutes. When a plurality of formulating agents are used, their degradation can each be over a controlled duration, these durations ranging for example over a total duration compatible with the residence time of the composition 10 in the body, and in particular in the gastrointestinal tract.

The released dihydrogen molecules can therefore be detected by the dihydrogen sensor 30, said sensor producing a signal which is transmitted by the wireless transmission device 31 to the device 33 external to the human or animal body 2. The dihydrogen sensor 30 and the wireless transmission device 31 are preferably connected to the wall of the stomach 22 of the human or animal body 2 by an anchor 32, as schematically illustrated in FIG. 4. The dihydrogen is diffused very rapidly into the medium constituted by the human or animal body 2, the ingestion of the composition 10 is detected in real time. Furthermore, the rapid diffusion of dihydrogen implies its low persistence at the site of its release and thus allows to discern potential variations in the amount of dihydrogen released.

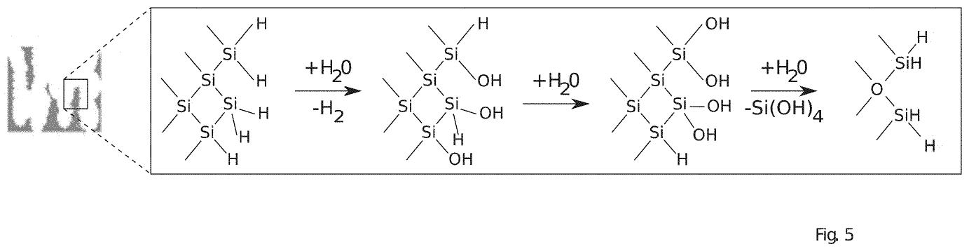

The invention therefore provides for storing the dihydrogen in a hydride 110 capable of dissolving upon contact with water. The amount of dihydrogen stored and which can be released is compatible with the intended application. Their abundance, their low cost, their ability to release a significant mass of dihydrogen (from 1 to 7.6% of dihydrogen released relative to the mass of product) and their non-toxicity make them prime candidates. A metal hydride is made up of metal atoms that constitute a host lattice for dihydrogen atoms trapped in interstitial sites, such as the metal surface or lattice defects. As illustrated in FIG. 5, a hydride 110 capable of dissolving upon contact with water preferably has, and in particular at its potential contact surface with a surrounding aqueous medium, a significant number of terminations or functional groups “—H” that are capable of spontaneously recombining with H₂O molecules by releasing molecular dihydrogen and by forming a passivating oxide layer on the surface of the hydride. Silicon, magnesium and calcium hydrides, which are in particular non-functionalised, are capable of giving rise to such recombinations.

Furthermore, the hydride 110 can be in different forms.

First, the hydride 110 can be porous in order in particular to increase the contact surface of the hydride with the surrounding aqueous medium and thus to increase the speed or equivalently the rate of release of the dihydrogen.

It is also considered to achieve a high speed and rate of dihydrogen release to use a powdered hydride. The hydride powder then preferably has particles 111 with an average size comprised between 10 nm and 10 μm. In addition, a combination of several hydrides, such as a calcium hydride and/or a titanium hydride and/or a magnesium hydride, and/or a combination of one or more dopants can be considered. For example, mechanical grinding of magnesium hydride with 20% calcium hydride for 10 hours allows the creation of defects on the surface of the hydride particles 111 and accelerates the speed of hydrolysis by 6.

A preferred embodiment of the invention is to use porous silicon as the hydride 110.

Methods for producing porous silicon, in particular reducible to powder, are known under the following names:
chemical dissolution (or “stain etching” which is described in particular in the article by DIMOVA-MALINOVSKA D., SENDO V A-VASSILEVA M., TZENOV N., KAMENOVA M., entitled “Preparation of thin porous silicon layers by stain etching” and published in Thin Solid Films, 1997, 297, pp. 9-12;
plasma etching (or “spark etching”) which is described in particular in the article by HUMMEL R. E., MORRONE A., LUDWIG M., CHANG S. S., entitled “On the origin of photoluminescence in the spark-eroded silicon” and published in J. Appl. Phys., 1993, 63, pp. 2771-2773; and
electrochemical anodisation which is described in particular in the article by SMITH R. L., COLLINS S. D., entitled “Porous silicon formation mechanisms” and published in J. Appl. Phys., 1992, 71, 8, pp. R1-R7 and the article by LEHMANN V., GOSELE U., entitled “Porous silicon formation: a quantum wire effect” and published in Appl. Phys. Lett., 1991, 58, pp. 856-858. The first two methods allow to produce porous silicon layers of the order of a few microns in thickness. Electrochemical anodisation, in turn, allows the production of thicker layers.

Whether the hydride 110 is made of porous silicon or otherwise, when it is in the form of a powder, its particles 111, each or on average, have a size comprised between 10 nm and 10 μm. The smaller the particle size, the more dihydrogen will be on board. For example, one molecule of silicon hydride, for example of formula SiH₄ alone will release two molecules of dihydrogen.

It may be desirable to have a lower speed and rate of dihydrogen release, in particular to modulate the release of dihydrogen for a longer determined duration of time. In this case, it will be preferable to use a non-porous hydride 110, which is in a volume form offering a contact surface limited by its shape with the aqueous medium intended to dissolve it.

Regardless of the composition or the form of the hydride 110, the dihydrogen that it stores is therefore formulated to be dispensed preferentially in the stomach 22 and in precise amounts using the water present in the human or animal body 2. It is indeed easily possible, in a composition 10 such as that introduced above, to finely control the amount of hydride 110 present in the composition 10. Said amount of hydride 110 is of course proportional to the amount of dihydrogen which will be released. In particular, it is possible to calculate the minimum amount of porous silicon, for example of the formula SiH₄, that can be detected in the stomach. Knowing that the stomach volume of a human body can reach 4 L, it is possible to detect 4 μg of dihydrogen, that is to say 2 μmol of dihydrogen. According to the dissolution reaction described in FIG. 5, this corresponds to 2 μmol of porous silicon of formula SiH₄, that is to say 64 μg. Taking into account a factor of 10 for the production efficiency of the hydride, and another factor of 10 for the detection limit of the dihydrogen sensor 30, it sufficient to have an amount of 6.4 mg of hydride 110 in the composition 10. This same calculation can be applied to modulate the amount of porous silicon in the composition 10 according to the desired amount of dihydrogen to be released. Note that this calculation can also be carried out for a calcium or magnesium hydride, for example of formula CaH₂ or MgH₂.

Controlling the amount of hydride 110 in the composition 10 therefore allows to finely control the amount of dihydrogen that will be released.

Compared to therapeutic compliance techniques, the present invention not only allows the detection of the ingestion of an active ingredient, but also the modulation of the amount of dihydrogen released in order to obtain a multitude of signatures potentially specific to a class of active ingredients, a particular active ingredient, or else a specific formulation of an active ingredient.

The embodiments of the composition 10 allowing said modulation to suppress the synthesis of numerous markers of different formulas and chemical properties. According to the present invention, the formulation of at least one hydride allows to obtain a multitude of signatures. Furthermore, the minimum amount of hydride allowing detection is relatively low. These embodiments are compatible with an industrial production by galenic techniques for wide deployment with a reduced production cost.

Another advantage of the composition 10 is that the signatures obtainable are based on at least one common detectable effect, the release of dihydrogen. Thus, a single dihydrogen sensor, preferably in the stomach, is necessary to measure the therapeutic compliance with the ingestion of many active ingredients. This advantageously allows to limit the equipment and in fact to limit the cost of the method for measuring the compliance.

In addition, the compliance measurement method is thus compatible with the accumulation of several treatments with different active ingredients for the same patient. Said method therefore advantageously provides easier and soothing compliance for the patient who no longer runs the risk of forgetting to take medication.

Compliance and adherence to therapy are thus promoted by the influence of said method on the patient's behaviour with respect to his treatment and its effects.

Other advantages will emerge from the description which is given below of different embodiments of the composition 10 as well as of the elements which can be used in the method for measuring the compliance.

FIGS. 1A to 1E schematically illustrate different embodiments of the composition 10.

FIG. 1A illustrates an embodiment of the composition 10 wherein the hydride 110 or the hydride particles 111 and the active ingredient 13 are mixed, potentially with a formulating agent 122, in a water-soluble tablet which can further be encapsulated by a formulating agent 121. In the example illustrated, the dissolution of the tablet, preferably in the stomach 22, results in the simultaneous release of the dihydrogen and of the active ingredient 13.

FIG. 1B illustrates an alternative embodiment of the composition 10 wherein the detection agent 11 comprising the hydride 110 or the hydride particles 111 coats the active ingredient 13. Each layer of the composition 10 according to this embodiment may further comprise encapsulation by a formulating agent 121. In the example illustrated, the degradation of the detection agent leads to the dissolution of the hydride 110, preferably in the stomach 22, and the release of the dihydrogen. Depending on the nature of the formulating agent 12 of the active ingredient 13, the delivery of the active ingredient 13 can be targeted to another part of the human or animal body 2, for example at the intestine 24 in the case where a gastro-resistant coating 121 is used and can dissolve at a pH characteristic of the intestine 24.

FIG. 1C illustrates a variant of the embodiment described by FIG. 1B, where the element comprising the active ingredient 13 is a capsule 15.

FIG. 1D illustrates an alternative embodiment of the composition 10, the sectional view of which along section 4 is shown in FIG. 1E. According to this embodiment, the detection agent 11 comprising the hydride 110 or the hydride particles 11 is coated with a layer comprising the active ingredient 13. Each layer of the composition 10 according to this embodiment comprises an encapsulation by a formulating agent 121. In the example illustrated, the degradation of the formulating agent 12 of the active ingredient 13 can take place in the mouth 21 for delivery of the active ingredient 13 into the mouth 21 or the oesophagus 23. Secondly, the degradation of the detection agent leads to the dissolution of the hydride 110, preferably in the stomach 22, and the release of the dihydrogen.

FIGS. 2A to 2E illustrate different embodiments of the composition 10, and more particularly different arrangements of one or a plurality of formulating agents 12 of said at least one detection agent 11. FIGS. 3A to 3E illustrate different modes of release of dihydrogen as a function of time t and thus various detectable signatures which may correspond to the embodiments illustrated by FIGS. 2A to 2E. These embodiments are described for the case where the different arrangements are disposed around a tablet or a capsule comprising the active ingredient 13. It should be noted that these embodiments may also be suitable for cases where the detection agent and the active ingredient 13 are mixed in a water-soluble tablet, as well as in cases where the detection agent is coated with the active ingredient 13.

FIG. 2A illustrates one embodiment of the composition 10 wherein the formulating agent 12 and the detection agent 11 are configured such that the distribution of the hydride 110 or the hydride particles 111 in the binder 122 is uniform. Thus, the release of dihydrogen occurs continuously and uniformly during the degradation of the detection agent 11, as illustrated in FIG. 3A.

FIG. 2B illustrates a variant of this embodiment of the composition 10 wherein the formulating agent 12 and the detection agent 11 are configured such that the distribution of the hydride 110 or the hydride particles 111 follows a concentration gradient, for example in a direction perpendicular to the main direction of extension of a layer of the composition. For example, the distribution of the hydride 110 or the hydride particles 111 in the binder 122 is increasing. Thus, the release of dihydrogen occurs continuously and increasingly during the degradation of the formulating agent of the detection agent 11, as illustrated in FIG. 3B. It should be noted that the case where the distribution of the hydride 110 or the hydride particles 111 in the binder 122 is decreasing is also possible.

FIG. 2C illustrates an embodiment of the composition 10 wherein a plurality of formulating agents 12 of said at least one detection agent 11 are configured so as to form a plurality of layers juxtaposed with at least one formulating agent 12 free of hydride. Thus, during the degradation of the detection agent 11, the release of dihydrogen takes place discontinuously over time, as illustrated in FIG. 3C.

FIG. 2D illustrates an embodiment of the composition 10 wherein a plurality of formulating agents 12 of said at least one detection agent 11 are configured so as to form a plurality of juxtaposed layers where the concentration of hydride 110 and/or the porosity of the hydride 110 are variable according to the layers. Thus, during the degradation of the detection agent 11, the amount of dihydrogen released varies over time, as illustrated in FIG. 3D.

The embodiments are not limited to the case described above but can moreover be combined with one another to obtain a multitude of modes of release of the dihydrogen. In particular, a plurality of active ingredients 13 and detection agents 11 can be configured together. As described in FIG. 2E, a first active ingredient 130 is intended to be delivered into a first specific part of the human or animal body 2, such as the mouth 21 or the oesophagus 23. The formulating agent 12 of the formulating detection agent 11 is intended to be degraded in a second specific part of the human or animal body 2, preferably in the stomach 22. A second active ingredient 131, for example contained in a gastro-resistant capsule, is intended to be delivered in a third specific part of the human or animal body 2, such as the intestine 24.

In addition, as described in FIG. 2E, the thicknesses of the formulating agents 12 of said at least one detection agent 11 and of said at least one active ingredient 13 are adjustable. Thus, the composition 10 according to this embodiment allows a release of dihydrogen in determined amounts over variable durations of time, as illustrated in FIG. 3E. The formulation of formulating agents 12 is also adaptable in order to modulate their degradation speed. For example, the degradation of formulating agents 12 comprising hydride particles 1110 and 1111 of different porosities induces a more or less rapid release of dihydrogen, as illustrated by the differences in the slopes of the curve illustrating the amount of dihydrogen released as a function of time in FIG. 3E.

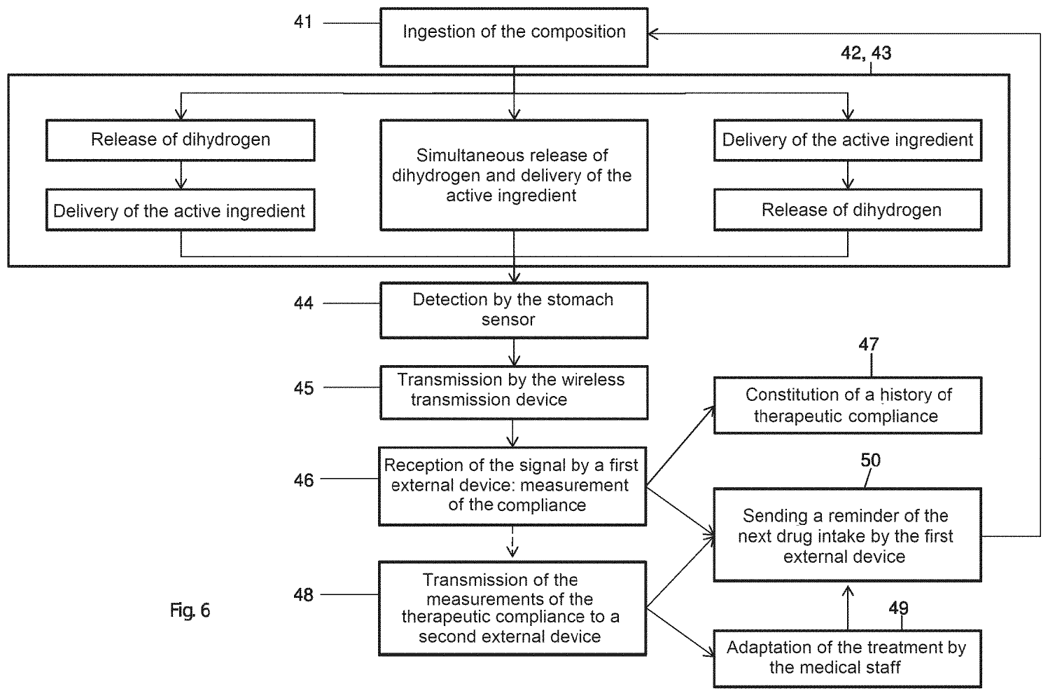

Referring to FIG. 6, the invention relates in a second aspect to a method for measuring therapeutic compliance implementing a measurement and communication system. The method is not limited to the steps illustrated in FIG. 6 and may comprise different arrangements of said steps as well as additional steps.

After the ingestion 41 of a composition 10 as described above, at least one step 42 of delivering the active ingredient 13 and at least one step 43 of releasing gas and/or dissolved dihydrogen, caused by the dissolution of hydride 110 and optionally degradation of formulating agent 12, are carried out.

According to the embodiment of the composition 10 illustrated in FIG. 1A, the delivery of the active ingredient 13 can be simultaneous with the release of the dihydrogen. The therapeutic compliance can thus be measured according to the method of the present invention, in the case of treatments where the active ingredient 13 is delivered into the stomach 22 of the patient, whether the therapeutic target is the stomach 22 or any other part of the human or animal body 2 reachable by the active ingredient 13 following its delivery.

According to the embodiment of the composition 10 illustrated in FIGS. 1B and 1C, the delivery of the active ingredient 13 may be subsequent to the release of the dihydrogen. The therapeutic compliance can thus be measured according to the method of the present invention, in the case of treatments requiring, for example, delivery of the active ingredient 13 to the intestine 24.

According to the embodiment of the composition 10 illustrated in FIGS. 1D and 1E, the delivery of the active ingredient 13 may be prior to the release of the dihydrogen. The therapeutic compliance can thus be measured according to the method of the present invention, in the case of treatments for example requiring delivery of the active ingredient 13 to the mouth 21 or the oesophagus 23.

The method for measuring therapeutic compliance comprises a step 44 of detecting the release of dihydrogen according to the methods described above. The released dihydrogen is detected, or else the amount of dihydrogen, and more particularly the change over time of this amount is measured by the dihydrogen sensor 30, said sensor preferably being in the stomach.

The method comprises a step 45 of transmitting a radiofrequency signal signifying the detection or else the measurement of the amount of dihydrogen released. This transmission is performed by the wireless transmission device 31 to at least one external device 33, for example by a Bluetooth Low Energy protocol in the example of a wireless transmission device in document FR 3059558 A1.

The method comprises the reception 46 or even the recording of the signal by a first external device 33, such as a smartphone, a digital tablet, a connected watch, a dedicated apparatus or else a module integrated or connected to a mobile phone or a digital tablet, allows to constitute 47 a history of therapeutic compliance of at least one treatment. Since a multitude of signatures can be detected by the measurement and communication system, the measurement of compliance with multiple treatments in parallel is also possible according to the embodiments described above. The patient can thus consult said history.

At least one second external device 34 can be added to the method, for example a server or a computer located in the patient's home or in a doctors office. The method can then comprise a step 48 of transmitting therapeutic compliance measurements from the first external device 33 to the second external device 34, for example via an internet or mobile connection. Said measurements can then be recorded by the second external device 34. In this way, the history of compliance with one or more treatments is also available to the medical staff. The medical staff can advantageously adapt 49 the treatment, for example adapt the dosage and follow the patient within the framework of personalised medicine.

The method may further comprise a step 50 of reminding the ingestion 41 of the composition. A reminder signal is then emitted at a precise moment by the external device 33, such as the patient's telephone, for the ingestion of the composition 10. The reminder signal is preferably a sound, light signal, a vibration or a written message. Signal emission is defined temporally relative to the initial or previous ingestion of the composition 10 or defined at specific times. If the ingestion of the composition 10 is not detected within a given time after the reminder, at least a second signal may be emitted by the external device 33.

Since the medical staff can adapt the treatment according to the measurements of therapeutic compliance, the medical staff can advantageously modify 49 the dosage of treatment. For this purpose, the medical staff can send via an internet or mobile connection new instructions by the external device 34, for example located in a doctors office, to an external device 33 or 34 which can be consulted by the patient in order, for example, to modify the parameters of step 50 of recalling the ingestion 41 of the composition.

The present invention finds a particularly advantageous application in measuring therapeutic compliance for any treatment, including in particular treatments for chronic diseases. In particular, its use for at least the compliance with anticoagulant treatments, against cardiac arrhythmias, with antihistamines or vitamin K antagonist or treatments sensitive to chronopharmacology, is considered.

The invention is not limited to the embodiments described above and extends to all the embodiments covered by the claims.

In particular, it is possible that the composition 10 further comprises an additional detection agent, different from that comprising at least one hydride 110. This additional detection agent would be, like that comprising at least one hydride 110, capable of allowing to indicate the ingestion of the drug substance 13 and in a way capable of measuring therapeutic compliance, but having a chemical nature that differentiates it from a hydride. More particularly, it can be bicarbonate. At least in contact with an aqueous medium in the human or animal body 2, the dissolution of bicarbonate is induced which leads to the release of carbon dioxide (CO₂). The carbon dioxide thus released is detectable by a carbon dioxide sensor. The latter can be comprised, where appropriate, in the measurement and communication system described above and interact with the communicating components of this system in the same way as the dihydrogen sensor. The carbon dioxide sensor can moreover be disposed in an ad hoc manner in the human or animal body, for example in the same way as the dihydrogen sensor is disposed therein.

The composition 10 according to this last feature can have any of the structures described above comprising a structuring of several hydrides as detection agents allowing detections which are de-correlated with one another over time. For example, composition 10 may comprise an onion structure coating which provides a bar code relating to the swallowed drug. This coating can be designed to generate a specific dissolution sequence allowing detection of drug intake.

It is thus possible to consider a sequence comprising a mixture of dihydrogen and carbon dioxide and/or a specific sequence involving these different gases.

An onion structure can for example comprise successively concentrically superimposed:
a first layer comprising hydride at a first concentration,
a second layer comprising bicarbonate at a first concentration,
a third layer comprising hydride at a second concentration, and
a fourth layer comprising bicarbonate at a second concentration.

A complex bar code signature is thus obtained corresponding to the drug intake.

1. A composition comprising:
a drug substance, selected from the group consisting of an active ingredient and a placebo, and
a detection agent,
wherein the drug substance is different than the detection agent, the composition being configured such that the detection agent allows to indicate an ingestion of the drug substance and allows to measure a therapeutic compliance,
wherein the detection agent comprises a hydride that will dissolve upon contact with an aqueous medium in a human or non-human animal body, thereby releasing dihydrogen into the aqueous medium.

2. The composition according to claim 1, wherein the hydride is formulated so as to release dihydrogen into the aqueous medium found at a specific part of the human or non-human animal body.

3. The composition according to claim 2, wherein the detection agent further comprises a bicarbonate that releases carbon dioxide into the aqueous medium found at the specific part of the human or non-human animal body.

4. The composition according to claim 1, further comprising a formulating agent in combination with the detection agent that will be degraded in a specific part of the human or non-human animal body, thereby releasing the detection agent.

5. The composition according to claim 4, wherein the formulating agent comprises at least one of:
a coating provided on the detection agent and
a binder holding a plurality of particles of the detection agent.

6. The composition according to claim 1, wherein the drug substance is mixed or juxtaposed with the detection agent.

7. The composition according to claim 1, further comprising a formulating agent in combination with the detection agent delaying a release of the detection agent and a delivery of the drug substance.

8. The composition according to claim 1, further comprising a formulating agent in combination with the detection agent providing a modulated release of dihydrogen from the detection agent.

9. The composition according to claim 1, the composition comprising a formulating agent in combination with the detection agent, wherein a distribution of the detection agent within the composition is non-uniform.

10. The composition according to claim 1, comprising a plurality of formulating agents in combination with the detection agent, configured relative to each other by being:
combined in a mixture,
arranged in concentric layers, or
arranged in a sequence of flat layers.

11. The composition according to claim 1, wherein the detection agent is porous.

12. The composition according to claim 1, wherein the detection agent is in the form of a powder.

13. The composition according to claim 1, wherein the hydride is selected from the group consisting of silicon hydride, magnesium hydride, calcium hydride, and mixtures thereof.

14. The composition according to claim 1, wherein the hydride comprises porous silicon.

15. A method for measuring therapeutic compliance, the method communication system, the system comprising:
deploying a dihydrogen sensor in the human body or non-human animal body,
deploying a wireless transmission device in the human body or non-human animal body, the wireless transmission device being operatively connected to the dihydrogen sensor,
arranging an external wireless reception device to receive signals from the wireless transmission device,
wherein, after a schedule administration of a composition according to claim 1 to the human body or non-human animal body,
detecting a level of dihydrogen at the dihydrogen sensor,
transmitting a signal from the wireless transmission device signifying that dihydrogen has been detected by the dihydrogen sensor, and
receiving the signal at the external wireless reception device indicating therapeutic compliance.

16. The method for measuring therapeutic compliance according claim 15, wherein the dihydrogen sensor is placed in a specific part of the human or non-human animal body.

17. The method for measuring the compliance according to claim 16, wherein the dihydrogen sensor is configured to provide a qualitative measure of the amount of dihydrogen detected at the dihydrogen sensor.

18. The method for measuring the compliance according to claim 15, further comprising:
reminding a patient to administer a prescribed dose of the composition according to claim 1 by emitting a reminder signal detectable by the patient after a predetermined period has elapsed from a preceding administration of the composition according to claim 1.

19. The composition according to claim 12, wherein the detection agent is in the form of a powder comprising particles having an average size ranging from 10 nm to 10 μm.

20. The method for measuring the compliance according to claim 16, wherein the dihydrogen sensor is placed in the stomach of the human or non-human animal body.
